(19) United States
(12) Patent Application Publication
(10) Pub. No.: US 2024/0395416 A1
(43) Pub. Date: Nov. 28, 2024

(54) System and Method for Generating a Patient Treatment Plan

(71) Applicant: David Shusterman, Hampton Bays, NY (US)

(72) Inventor: David Shusterman, Hampton Bays, NY (US)

(21) Appl. No.: 18/201,716

(22) Filed: May 24, 2023

(51) Int. Cl. G16H 50/70

(52) U.S. Cl. CPC G16H 50/70; G16H 10/20; G16H 10/40; G16H 50/20

(57) ABSTRACT

Systems and methods for generating an efficient patient treatment plan are disclosed. An example system includes a user interface, a planning engine, a learning interface, and a planning engine reconfiguration routine. The user interface is configured to receive patient information associated with a current patient and to provide information indicative of an efficient treatment plan. The planning engine is responsive to said patient information and configured to provide a portion of a treatment plan based on said patient information. The learning interface is configured to receive learning information indicative of a resultant efficiency of said portion of said treatment plan. The planning engine reconfiguration routine is operative to adjust said planning engine based on said learning information.

### BACKGROUND OF THE INVENTION

### Field of the Invention

This invention relates generally to healthcare, and more particularly to patient treatment planning.

### Description of the Background Art

Healthcare facilities currently employ computer-based patient treatment planning systems. Such systems aid physicians in providing patients with treatment plans including drug regimens, physical regimens, specialist referrals, etc. One challenge with such systems is that they often incorrectly refer patients to specialists. As a result, many of the patients referred to specialists are turned back to their original physicians during the initial appointment without any type of treatment ever taking place. Of course, such inefficiency wastes the time and money of both the patient and the specialist involved.

### SUMMARY

The present invention overcomes the problems associated with the prior art by providing an intelligent patient referral system. Aspects of the invention facilitate the development of more efficient patient treatment plans.

An example method for providing an efficient patient treatment plan includes receiving first patient information associated with a first patient, and using the first patient information to generate a portion of a treatment plan according to predetermined criteria. The example method additionally includes receiving information indicative of the efficacy of the portion of the treatment plan, and using the information indicative of the efficacy of the portion of the treatment plan to modify the predetermined criteria to generate a second predetermined criteria. The example method additionally includes receiving second patient information associated with a second patient, and using the second patient information to generate another portion of a treatment plan according to the second predetermined criteria.

In an example method, the first patient information can be obtained from historical medical records. The information indicative of the efficacy of the portion of the treatment plan can be generated using the historical records. The historical records can include information indicative of the efficiency of a completed treatment plan of the first patient including the portion of the treatment plan.

In a particular example method, the second patient information can include a prostate volume, and the second predetermined criteria can include a prostate volume threshold. The second patient information can also include a prostate specific antigen (PSA) level, and the second predetermined criteria can include a PSA threshold value.

In another particular example method, the second patient information can include a patient survey score, and the second predetermined criteria can include at least one particular survey score value. The patient survey score can be based on answers from the second patient related to the functioning of the genitourinary system of the second patient. For example, the patient survey score can be obtained at least partially based on questions relating to the frequency of urination. As another example, the patient survey score can be obtained at least partially based on questions relating to the urgency of urination. As yet another example, the patient survey score can be obtained at least partially based on questions relating to difficulty of urination. As yet another example, the patient survey score can be obtained at least partially based on questions relating to the flow rate of urination. Optionally, the patient survey score can be obtained at least partially based on any combination of questions relating to the frequency of urination, the urgency of urination, difficulty of urination, and/or flow rate of urination.

Example systems for generating efficient patient treatment plans are also disclosed. One example system includes a user interface, a planning engine, a learning interface, and a planning engine reconfiguration routine. The user interface can be configured to receive patient information associated with a current patient and to provide information indicative of an efficient treatment plan. The planning engine can be responsive to the patient information and configured to provide a portion of a treatment plan based on the patient information. The learning interface can be configured to receive learning information indicative of a resultant efficiency of the portion of the treatment plan. The planning engine reconfiguration routine can be operative to adjust the planning engine based on the learning information.

In an example system, the learning information can be derived from historical medical records. The learning information can include prior patient information, a treatment plan followed by the prior patient, and information indicative of the efficiency of the treatment plan followed by the prior patient.

In a particular example system, the patient information associated with the current patient can include a prostate volume. The planning engine can use a prostate volume threshold to generate the portion of the treatment plan. As another example, the current patient information can include a prostate specific antigen (PSA) level, and the planning engine can use a PSA threshold value to generate the portion of the treatment plan.

In a particular example system, the patient information associated with the current patient can include a patient survey score. The planning engine can use at least one particular survey score value to generate the portion of the treatment plan. The patient survey score can be based on answers from the current patient related to the functioning of the genitourinary system of the current patient. For example, the patient survey score can be obtained at least partially based on questions relating to the frequency of urination. As another example, the patient survey score can be obtained at least partially based on questions relating to the urgency of urination. As yet another example, the patient survey score can be obtained at least partially based on questions relating to difficulty of urination. As yet another example, the patient survey score can be obtained at least partially based on questions relating to the flow rate of urination. Optionally, the patient survey score can be obtained at least partially based on any combination of questions relating to the frequency of urination, the urgency of urination, difficulty of urination and/or a flow rate of urination.

### BRIEF DESCRIPTION OF THE DRAWINGS

The present invention is described with reference to the following drawings, wherein like reference numbers denote substantially similar elements:

FIG. 1 is a block diagram of an internetwork between primary care facilities, specialist facilities, testing/diagnostic facilities, a patient record database, and a historic record database, all in communication with a health care planning system via an internetwork;

FIG. 2 is a block diagram showing a server of the health care planning system of FIG. 1;

FIG. 3 is a flowchart summarizing an example method (e.g., a questionnaire) for acquiring patient information;

FIG. 4A is a flowchart summarizing an example method for generating a portion of a patient treatment plan using the information acquired by the method/questionnaire of FIG. 3;

FIG. 4B is a flowchart summarizing an example method for generating another portion of the patient treatment plan using the information acquired by the method/questionnaire of FIG. 3;

FIG. 5 is another flowchart for generating a patient treatment plan according to the information acquired by the method/questionnaire of FIG. 3;

FIG. 6 is a block diagram illustrating the training of a machine learning system for generating a patient treatment plan with historical data;

FIG. 7 is a block diagram illustrating the use and training the machine learning system of FIG. 6 in real-time; and

FIG. 8 is a flowchart summarizing an example method for generating an efficient patient treatment plan.

### DETAILED DESCRIPTION

The present invention overcomes the problems associated with the prior art, by providing a system and method that generates a treatment plan based in-part on historical health records of multiple patients. In the following description, numerous specific details are set forth (e.g., type of specialists, type of treatments, etc.) in order to provide a thorough understanding of the invention. Those skilled in the art will recognize, however, that the invention may be practiced apart from these specific details. In other instances, details of well-known computing practices (e.g., machine learning, database structures, etc.) and components have been omitted, so as not to unnecessarily obscure the present invention.

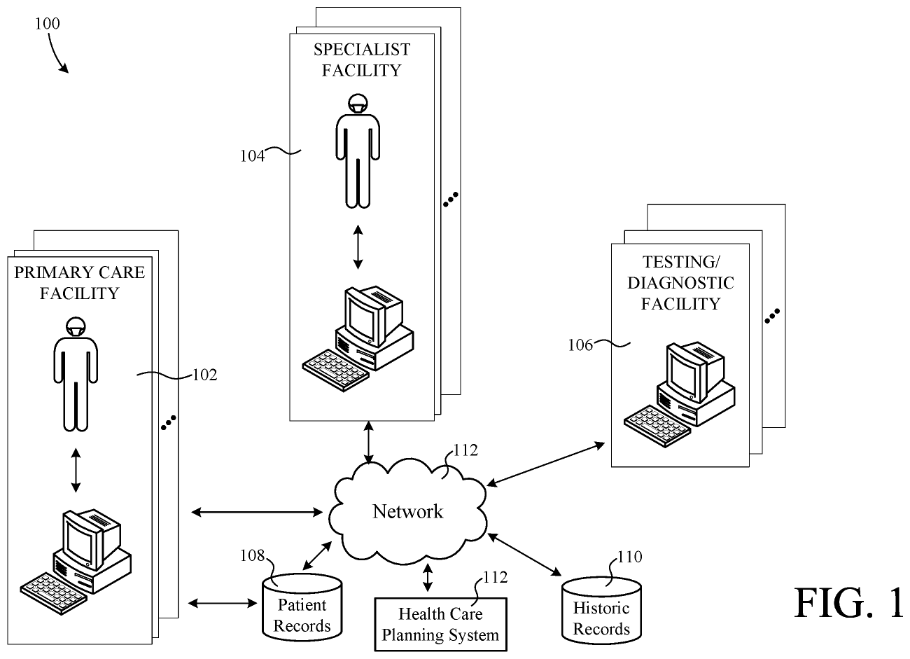

FIG. 1 is a block diagram showing a system 100 for generating efficient patient treatment plans. System 100 includes a plurality of primary care facilities 102, a plurality of specialist facilities 104, a plurality of testing/diagnostic facilities 106, a patient record database 108, a historic records database 110, and a health care planning system 112, all interconnected via an internetwork 114 (e.g., the Internet).

Primary care facilities 102 include, for example, general medical practitioner offices. Primary care facilities 102 may also include, but are not limited to, hospitals, mental health facilities, dental offices, and so on.

Specialist facilities 104 include, for example, urology clinics, cardiology clinics, neurology clinics, and so on. However, specialist facilities 104 may also include various other types of specialist facilities including, but not limited to, orthopedic clinics, dermatology clinics, mental health clinics, clinics, oral surgery clinics, and any other type of specialist facility to which patients are commonly referred by primary care facilities.

Testing/diagnostic facilities 106 can include any third party facility used for specialized medical testing and/or diagnostics. Examples of such testing/diagnostic facilities include, but are not limited to, laboratories, radiology facilities, ultrasound facilities, and so on.

Patient records database 108 includes medical records of patients associated with one or more primary care facilities. Patient records database 108 may include one or more remote databases accessible to primary care facilities 102 indirectly through network 112 and/or may include one or more local databases directly accessible within primary care facilities 102.

Historic records database 110 includes information associated with past patient courses of treatment and the resulting outcomes. For example, historic records database 110 may include a patient treatment plan that instructed a patient to take a particular medication and how effective the medication was, specialist referrals, diagnostic tests, and so on. Such records may be acquired from primary care facilities 102, specialist facilities 104, testing/diagnostic facilities 106, patient database 108, and/or any other source of records relating to patient treatments and outcomes.

Health care planning system 112 is configured to generate a patient treatment plan based at least in part on information acquired directly from the patient (e.g., physical exam, patient questionnaire, etc.), from patient records database 108, and/or historic records database 110. Although health care planning system 112 is shown as a remote system accessible through network 112, health care planning system 112 may also be located partially or completely in any one or more of primary care facilities 102, specialty facilities 104, and/or testing/diagnostic facilities 106.

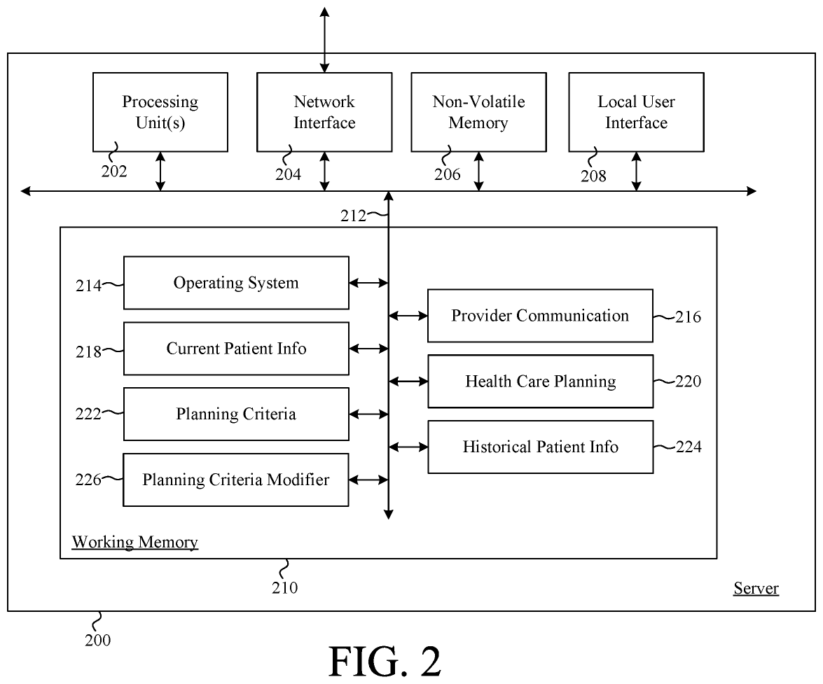

FIG. 2 is a block diagram of a server 200 of health care planning system 112, which is configured to generate patient treatment plans. Server 200 is connected to network 112 and includes one or more processing unit(s) 202, a network interface 204, non-volatile memory 206, a local user interface 208, and a working memory 210, all interconnected by a system bus 212, which facilitates intercommunication between the various components of server 200.

Processing unit(s) 202 execute data and code contained in working memory 210 to cause server 200 to carry out its intended functions (e.g. generate patient treatment plans). For illustrative purposes, subsets of code are represented in working memory 210 as functional blocks. However, this is by way of example and explanation only. The present invention is not limited by any particular arrangement or structure of the computer code, unless explicitly set forth in the appended claims.

Server 200 communicates over network 112 via network interface 204. Network interface 204 transmits data packets onto and receives data packets from internetwork 112, thus allowing server 200 to communicate with primary care facilities 102, specialist facilities 104, testing/diagnostic facilities 106, patient records database 108, and historic records database 110.

Non-volatile memory 206 (e.g., read-only memory, hard drive(s), etc.) provides storage for data and code (e.g., boot code and programs) that are retained even when server 200 is powered down.

Local user interface 208 facilitates communications local users and includes, by way of nonlimiting example, a keyboard, a mouse, a monitor, a printer, and other such devices that facilitate communications between server 200 and a user and/or administrator.

Working memory 210 (e.g. random access memory) provides dynamic memory to server 200, and can store executable code (e.g. an operating system 214), which is loaded into working memory 210 during system start-up. Operating system 214 facilitates control and coordination of other modules loaded into working memory 210.

Working memory 210 further includes a provider communication module 216, current patient information 218, a health care planning module 220, planning criteria 222, historical patient information 224, and a planning criteria modifier module 226. The various modules and data are initialized and loaded into working memory 210 at startup from non-volatile memory 206 using methods well known to those skilled in the art. Communication module 216 facilitates communication between server 200 and any one or more of primary care facilities 102, specialist facilities 104, testing/diagnostic facilities 106, patient records database 108, and historic records database 110.

Current patient information 218 includes information associated with a patient that is currently seeking a treatment plan. Such information may include, without limitation, results of a physical examination, diagnostic results, answers to a questionnaire, and/or general information about the patient such as, for example, age, sex, weight, height, blood pressure, etc. Health care planning module 220 generates treatment plan(s) for the patient based at least in-part on current patient information 218 and planning criteria 222. Planning criteria 222 includes predetermined criteria for generating the patient treatment plan based on patient information 218. For example, one predetermined criteria may be a specific prostate volume that when exceeded causes health care planning module 220 to generate a patient treatment plan that includes referring the patient to a specialist (e.g. a Urologist).

Historical patient information 224 includes historical patient information from, for example, historic records database 110. This historical patient information may include past treatment plans and outcomes resulting therefrom associated with a number of other past patients. For example, such historical patient information may include a treatment plan that referred a patient to a urologist (e.g., because his prostate size exceeded a specific volume), but where the ultimate successful therapy could have been provided by the general practitioner, thereby avoiding an unnecessary visit to the urologist. In other words, a referral to a specialist was not justified by the planning criteria. In general, historical patient information can include a great multitude of examples of efficient and inefficient treatment plans for past patients associated with particular patient information (e.g., the past patient's history, exam results, diagnostic results, and so on.

Planning criteria modifier 226 is configured to modify the planning criteria in planning criteria module 222, using historical patient information 224 as a guide, to improve the efficacy of health care plans generated by health care planning module 220. For example, planning criteria modifier module 226 may increase a prostate volume criterion, if the current volume criterion results in more unnecessary visits to a urologist than the updated criterion. As a result, subsequently generated patient treatment plans will be less likely to result in an unnecessary referral.

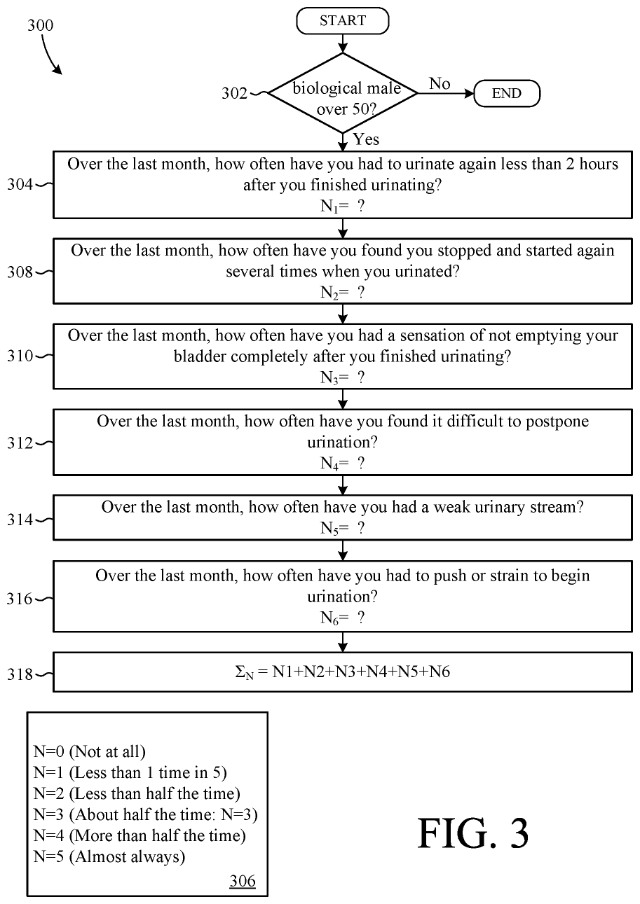

FIG. 3 is a flowchart summarizing an example method/questionnaire 300 used to obtain a score from a patient that can be used to generate a patient treatment plan. In a first step 302, it is determined if the patient is a biological male over the age of fifty. If not, method 300 ends. If so, then in a second step 304, the number of times within a month that the patient has had to urinate less than two hours after the previous urination is determined, a corresponding value is assigned according to a key 306, and the value is recorded as N₁. Then, in third step 308, the number of times within a month that the patient has stopped and restarted urination is determined, a corresponding value is assigned according to key 306, and the value is recorded as N₂. Next, in a fourth step 310, the number of times within a month that the patient has had a sensation of not completely emptying their bladder after urination is determined, a corresponding value is assigned according to key 306 and the value recorded as N₃. Then, in a fifth step 312, the number of times within a month that the patient has found it difficult to postpone urination is determined, a corresponding value is assigned according to key 306 and, and the value is recorded as N₄. Next, in a sixth step 314, the number of times within a month that the patient has had a weak urine stream is determined, a corresponding value is assigned according to key 306, and the value is recorded as N₅. Then, in a seventh step 316, the number of times within a month that the patient has had to push or strain to urinate is determined, a corresponding value is assigned according to key 306, and the value is recorded as N₆. Finally, in an eighth step 318, the summation of N₁+N₂+N₃+N₄+N₅+N₆ is determined and recorded as Σ_{N}.

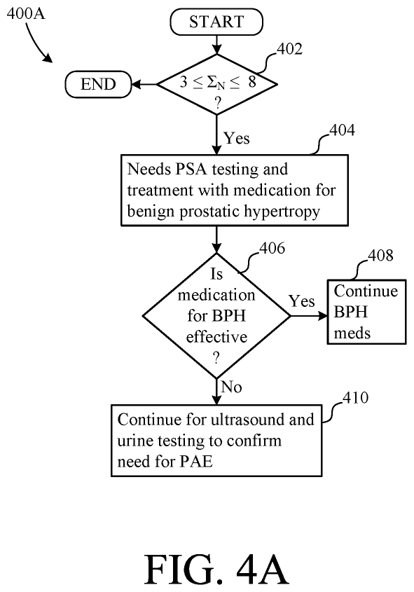

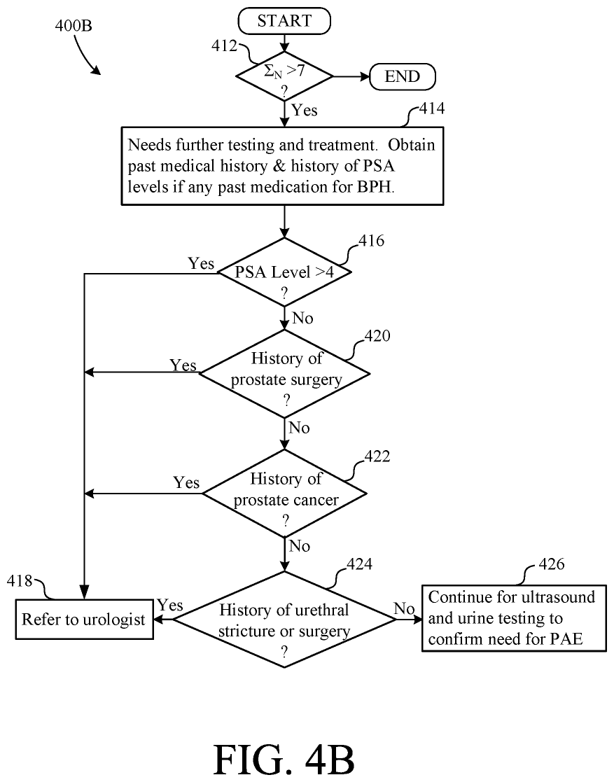

FIG. 4A is a flowchart summarizing an example method 400A for generating a first portion of a patient treatment plan using the input from method/questionnaire 300. In a first step 402, health care planning module 220 determines if 3≤Σ_{N}≤8. If not, 400A ends, and no recommended plan is generated. If 3≤Σ_{N}≤8, then in a second step 404, the portion of the patient treatment plan indicates that the patient needs prostate specific antigen (PSA) testing and treatment with medication for benign prostatic hypertrophy (BPH). Next, in a third step 406, it is determined if the medication for the BPH is effective. If so, in a fourth step 408, the patient treatment plan indicates that the patient should continue the BPH medication. If the BPH medication is not effective, then in a fifth step 410, the patient treatment plan instructs the patient to continue for ultrasound and urine testing to confirm the need for prostatic artery embolization (PAE).

FIG. 4B is a flowchart summarizing an example method 400B for generating a second portion of a patient treatment plan using the input (e.g., prostate score Σ_{N}) from method/questionnaire 300. In a first step 412, it is determined if Σ_{N}>7. If not, method 400B ends, and no additional treatment plan is recommended. If, in first step 412, it is determined that Σ_{N}>7, then in a second step 414, the second portion of the patient treatment plan will indicate that the patient needs further testing and treatment, and information related to past medical history, any history of PSA levels, and any past medication prescribed for BPH should be obtained. Next, in a third steep 416, it is determined if the patient's PSA level is greater than 4. If so, method 400B proceeds to a fourth step 418, and the second portion of the patient treatment plan will include a referral to a urologist. If the PSA level is not greater than 4, then, in a fifth step 420, it is determined if the patient has a history of prostate surgery. If, in step 420, it is determined that the patient does have a history of prostate surgery, method 400B proceeds to fourth step 418, and the second portion of the patient treatment plan will include a referral to a urologist. If, in step 420, it is determined that the patient does not have a history of prostate surgery, then method 400B proceeds to a sixth step 422, where it is determined if the patient has a history of prostate cancer. If it is determined that the patient does have a history of prostate cancer, method 400B proceeds to fourth step 418, and the second portion of the patient treatment plan will include a referral to a urologist. If, in sixth step 422, it is determined that the patient does not have a history of prostate cancer, then in a seventh step 424 it is determined if the patient has a history of urethral stricture or surgery. If the patient does have a history of urethral stricture or surgery, method 400B proceeds to fourth step 418, and the second portion of the patient treatment plan will include a referral to a urologist. If it is determined that the patient does not have a history of urethral stricture or surgery, then, in an eighth step 426, the second portion of the patient treatment plan will include a recommendation for ultrasound and urine testing to confirm the need for PAE.

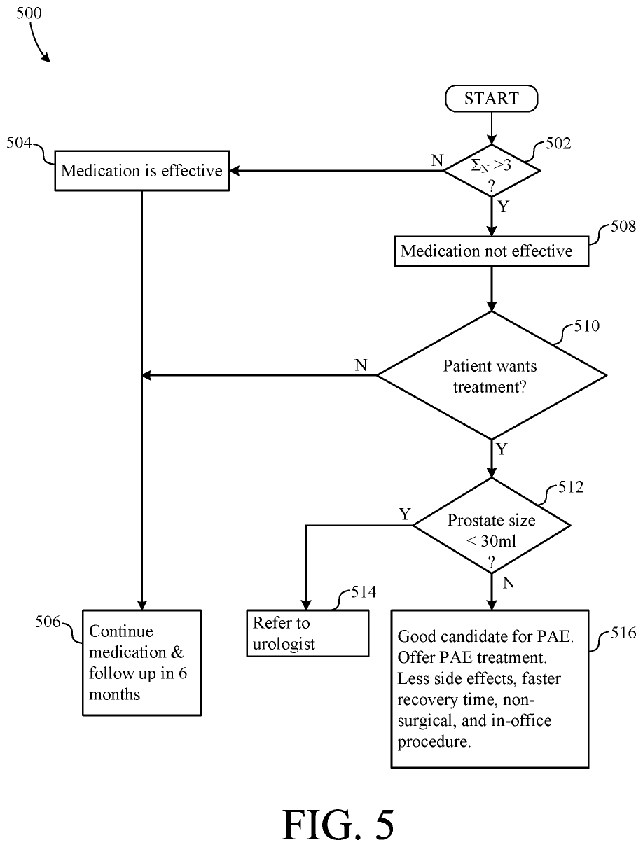

FIG. 5 is a flowchart summarizing a method 500 for generating a patient treatment plan following a period of use of BPH medication, using the input from questionnaire 300. In a first step 502, it is determined if the prostate score is greater than 3: Σ_{N}>3. If not, then in a second step 504, it is concluded that the medication is effective and, in a third step 506, the treatment plan includes instructing the patient to continue medication and follow up at some future time (e.g., in six months). If, in first step 502, it is determined that Σ_{N}>3, then in a fourth step 508, it is concluded that the medication is not effective. Next, in a fifth step 510, it is determined if the patient wants additional treatment. If not, then method 500 proceeds to third step 506, and the treatment plan will include instructing the patient to continue medication and follow up at a later date (e.g., in six months). If, however, it is determined in step 510 that the patient wants treatment, then, in a sixth step 512, it is determined if the volume of the patient's prostate is less than 30 ml. If so, then in a seventh step 514, the patient treatment plan will include a referral to a urologist. If it is determined in sixth step 512 that the volume of the patient's prostate is 30 ml or more, then in an eighth step 516, the patient treatment plan will indicate that the patient is a good candidate for PAE, the patient is offered PAE treatment, and the patient can be informed that the PAE treatment has less side effects, a faster recovery time, is non-surgical, and is an in-office procedure.

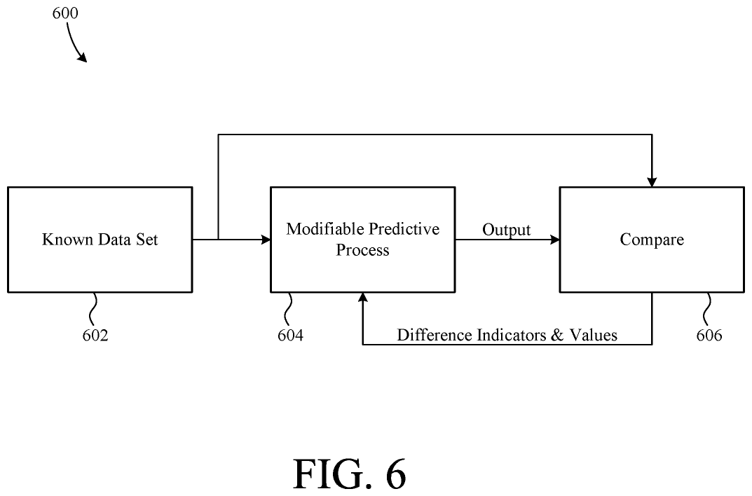

FIG. 6 is a block diagram 600 illustrating machine learning and pre-training of health care planning system 112. In training system 112, data from a known data set 602 is provided to a modifiable predictive process (MPP) 604 and a comparison process 606. The data set includes, for example, historical patient information (e.g., prostate size, age, lab results, and so on), provided treatments, and actual outcomes corresponding to that patient information. Modifiable predictive process 604 generates a treatment plan based on the provided patient information, and the generated treatment plan is output to comparison process 606. Comparison process 606 then compares the generated patient treatment plan with a known efficient and effective patient treatment plan and then outputs difference indicators and values back to modifiable predictive process 604. Modifiable predictive process 604 then adjusts weighted values within treatment plan generating algorithms to minimize the difference between the generated plan and the known effective and efficient treatment plan. This training process is repeated with extremely large data sets, until the patient treatment plans output from modifiable predictive process 604 accurately reflects the known efficient and effective patient treatment plans. This feedback loop may continue for as many iterations as needed until modifiable predictive process 604 can output accurate patient treatment plans. In other words, modifiable predictive process 604 is machine learning system that is pre-trained with known data before it is used to generate novel patient treatment plans for patients based on current patient information.

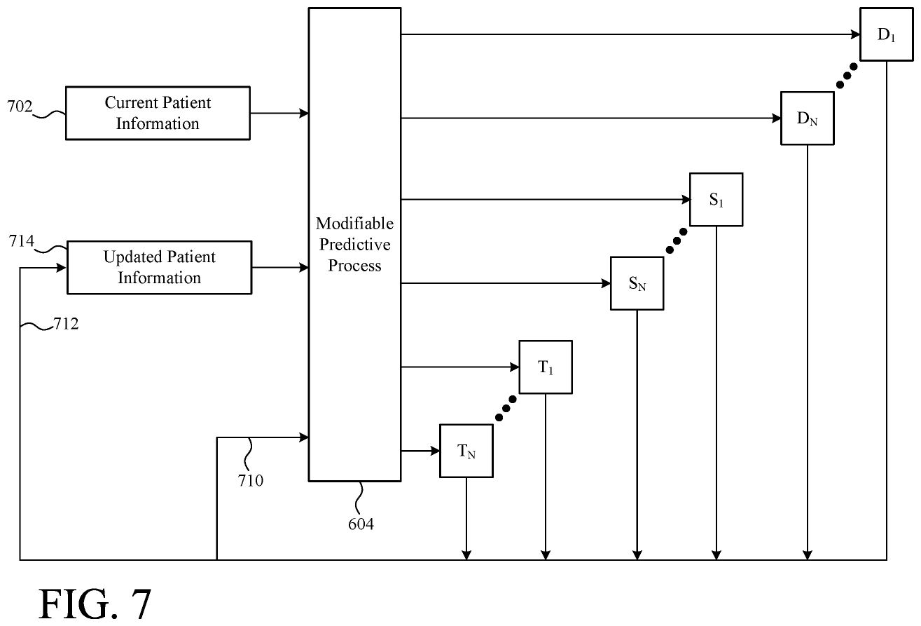

In addition to pre-training, planning system 112 can also learn in real-time from patient information associated with current patients following generated patient treatment plans. FIG. 7 is a block diagram illustrating ongoing, real-time machine learning and training of health care planning system 112. In system 112, modifiable predictive process 604 receives input, generates a portion of a patient treatment plan based on the input, receives feedback based on the implementation of the plan, and adjusts its predictive process ‘(“learns”) to generate a more accurate patient treatment plan from the feedback.

MPP 604 receives input in the form of current patient information 702. Current patient information 702 includes medical information associated with the current patient being treated, including for example, but not limited to, past medical history, family medical history, results of physical exams, diagnostic information, and so on.

Based on the current patient information, MPP 604 generates an initial portion of a treatment plan, which can include, but is not limited to, one or more diagnostic/testing requests (D₁-D_{N}), specialist referrals (S₁-S_{N}), and/or treatment plans (T₁-T_{N}). Diagnostic/testing requests (D₁-D_{N}) can include, by way of non-limiting example, blood tests, urine tests, imaging studies, biopsies, and so on. Specialist referrals (S₁-S_{N}) are patient referrals to medical specialists, which can include, by way of non-limiting example, referrals to urologists, cardiologists, dermatologists, or any other medical specialist. Treatment plans (T₁-T_{N}) can include, by way of non-limiting example, drug therapy, physical therapy, dietary restrictions, surgical intervention, and so on.

Any results of diagnostic/testing requests (D₁-D_{N}), specialist referrals (S₁-S_{N}), and treatment plans (T₁-T_{N}) are then fed back into MPP 604 through a learning input 710. The results are also provided back into the primary care facility, via a communication path 712, to update the current patient information to generate updated patient information 714. The updated patient information 714 can then be provided back into RPP 604 to generate a next portion of the treatment plan for the current patient. This process is continually repeated as many times as are necessary to resolve the current patient's medical issues.

This feedback received via learning input 710 is accumulated by MPP 604 and can be used to determine the effectiveness of each recommended portion of the patient treatment plan. Based on the determined efficiency and effectiveness of each portion of the generated patient treatment plans, the predictive processes of MPP 604 can be modified to generate ever more efficient and effective patient treatment plans as time goes on.

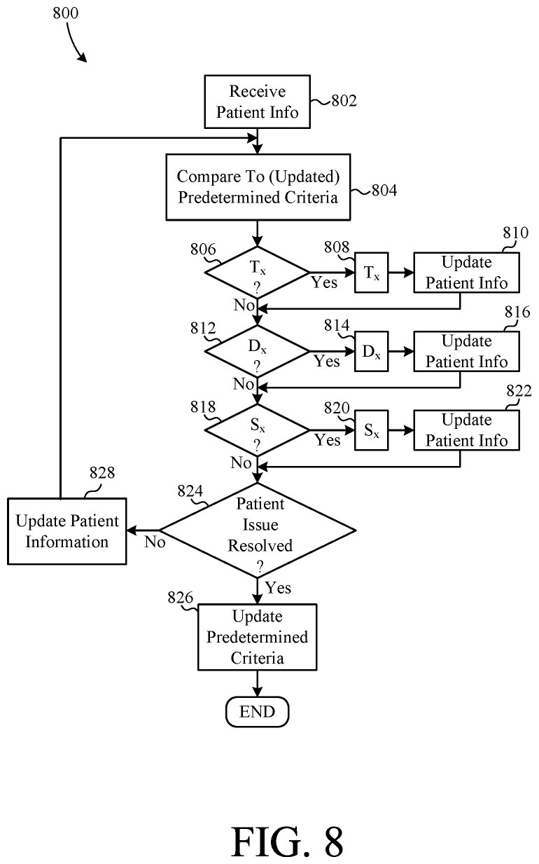

FIG. 8 is a flowchart summarizing a method 800 for generating an efficient patient treatment plan. In a first step 802, patient information is received. Then, in a second step 804, the received patient information is compared to predetermined criteria. Next, in a third step 806, it is decided whether any treatment is recommended. If so, then in a fourth step 808, the recommended treatment is provided. Then, in a fifth step 810, the patient information is updated to reflect the treatment provided in fourth step 808. Next, in a sixth step 812, it is decided if any diagnostic/testing is recommended. If so, the recommended diagnostic/testing is carried out in a seventh step 814. Then, in an eighth step 816, the patient information is updated to include information indicative of the results of the diagnostic/testing. Next, in a ninth step 818, it is determined if a referral to a specialist is recommended. If so, then in a tenth step 820, a specialist referral is completed. Then, in an eleventh step 822, the patient information is updated to include information indicative of the report of the specialist. Next, in a twelfth step 824, it is determined whether the patient's issue is resolved. If not, method 800 returns to second step 804, and method 800 is repeated, using the updated patient information to generate a next segment of the current patient treatment plan. If, however, it is determined in twelfth step 824 that the patient's issue is resolved, the predetermined criteria of step 804 can be updated in a step 826, based on any perceived inefficiencies and/or ineffectiveness of portions of in the overall patient treatment plan.

The description of particular embodiments of the present invention is now complete. Many of the described features may be substituted, altered or omitted without departing from the scope of the invention. For example, alternate questionnaires will be used for different specialist types (e.g., orthopedic surgeon, cardiologist, etc.), instead of the urology-based questionnaire. As another example, the invention may be implemented in other health care facilities outside of medical facilities such as dentist offices, chiropractic offices, etc. These and other deviations from the particular embodiments shown will be apparent to those skilled in the art, particularly in view of the foregoing disclosure.

We claim:

1. A method for providing an efficient patient treatment plan, said method including:
receiving first patient information associated with a first patient;
using said first patient information to generate a portion of a treatment plan according to predetermined criteria;
receiving information indicative of the efficacy of said portion of said treatment plan;
using said information indicative of said efficacy of said portion of said treatment plan to modify said predetermined criteria to generate a second predetermined criteria;
receiving second patient information associated with a second patient; and
using said second patient information to generate another portion of a treatment plan according to said second predetermined criteria.

2. The method of claim 1, wherein:
said first patient information is obtained from historical medical records; and
said information indicative of said efficacy of said portion of said treatment plan is generated using said historical records; and
said historical records include information indicative of the efficiency of a completed treatment plan of said first patient including said portion of said treatment plan.

3. The method of claim 1, wherein:
said second patient information includes a prostate volume; and
said second predetermined criteria includes a prostate volume threshold. 3

4. The method of claim 1, wherein:
said second patient information includes a patient survey score; and
said second predetermined criteria includes at least one particular survey score value.

5. The method of claim 4, wherein said patient survey score is based on answers from said second patient related to the functioning of the genitourinary system of said second patient.

6. The method of claim 5, wherein said patient survey score is obtained at least partially based on questions relating to the frequency of urination.

7. The method of claim 5, wherein said patient survey score is obtained at least partially based on questions relating to the urgency of urination.

8. The method of claim 5, wherein said patient survey score is obtained at least partially based on questions relating to difficulty of urination.

9. The method of claim 5, wherein said patient survey score is obtained at least partially based on questions relating to the flow rate of urination.

10. The method of claim 9, wherein said patient survey score is obtained at least partially based on questions relating to the frequency of urination, the urgency of urination, and difficulty of urination.

11. The method of claim 1, wherein:
said second patient information includes a prostate specific antigen (PSA) level; and
said second predetermined criteria includes a PSA threshold value.

12. A system for generating an efficient patient treatment plan, said system comprising:
a user interface configured to receive patient information associated with a current patient and to provide information indicative of an efficient treatment plan;
a planning engine responsive to said patient information and configured to provide a portion of a treatment plan based on said patient information;
a learning interface configured to receive learning information indicative of a resultant efficiency of said portion of said treatment plan; and
a planning engine reconfiguration routine operative to adjust said planning engine based on said learning information.

13. The system of claim 12, wherein said learning information is derived from historical medical records and includes:
prior patient information;
a treatment plan followed by said prior patient; and
information indicative of the efficiency of said treatment plan followed by said prior patient.

14. The system of claim 12, wherein:
said patient information associated with said current patient includes a prostate volume; and
said planning engine uses a prostate volume threshold to generate said portion of said treatment plan.

15. The system of claim 12, wherein:
said patient information associated with said current patient includes a patient survey score; and
said planning engine uses at least one particular survey score value to generate said portion of said treatment plan.

16. The system of claim 15, wherein said patient survey score is based on answers from said current patient related to the functioning of the genitourinary system of said current patient.

17. The system of claim 16, wherein said patient survey score is obtained at least partially based on questions relating to the frequency of urination.

18. The system of claim 16, wherein said patient survey score is obtained at least partially based on questions relating to the urgency of urination.

19. The system of claim 16, wherein said patient survey score is obtained at least partially based on questions relating to difficulty of urination.

20. The system of claim 16, wherein said patient survey score is obtained at least partially based on questions relating to the flow rate of urination.

21. The system of claim 20, wherein said patient survey score is obtained at least partially based on questions relating to the frequency of urination, the urgency of urination, and difficulty of urination.

22. The system of claim 12, wherein:
said current patient information includes a prostate specific antigen (PSA) level; and
said planning engine uses a PSA threshold value to generate said portion of said treatment plan.
